(19) United States
(12) Patent Application Publication
(10) Pub. No.: US 2011/0311562 A1
(43) Pub. Date: Dec. 22, 2011

(54) Glycolipid Mixture with Anti-Inflammatory Activity Obtained from Oscillatoria Planktothrix

(76) Inventor: Monica Molteni, Agrate Brianza (IT)

(21) Appl. No.: 13/201,967

(22) PCT Filed: Feb. 17, 2010
(86) PCT No.: PCT/EP2010/051958

§ 371 Date: Aug. 17, 2011

(30) Foreign Application Priority Data
Feb. 18, 2009 (IT) MI2009A000212

(51) Int. Cl. A61K 31/70; A61P 25/28; A61P 31/00; A61P 25/00; A61P 25/08; A61P 11/06; A61P 29/00; C12S 3/20; A61P 25/16

(52) U.S. Cl. 424/184.1; 424/283.1; 435/270

(57) ABSTRACT

It was observed that a highly purified preparation of polar glycolipids extracted from cells of the cyanobacterium Oscillatoria Planktothrix is characterized by the presence of at least one species of high molecular weight glycolipid comprising one or more units of rhamnose. In such mixture, having a level of nucleic acid contamination lower than (or equal to) 3%, an inhibitory activity toward ATP synthase (ATP-SX) was identified which is capable of decreasing the level of extracellular ATP and thereby the extent of the inflammatory response. The glycolipid mixture is especially useful in inflammation induced by ischemic stimuli, cancer or autoimmune diseases (such as multiple sclerosis, inflammatory bowel disease (Crohn's disease, ulcerative colitis), psoriasis, rheumatoid arthritis, diabetes, autoimmune thyroiditis, systemic lupus erythematosus), in addition to systemic inflammatory states such as systemic inflammatory syndrome (SIRS), sepsis, vasculitis, or in localized inflammatory states, such as neurodegenerative diseases or asthma.

### FIELD OF THE INVENTION

The present invention relates to a glycolipid mixture prepared from the cells of a cyanobacterium, wherein the main component is a rhamnose-containing high-molecular-weight glycolipid with anti-inflammatory and immunomodulatory properties.

### STATE OF THE ART

Cyanobacteria, also called blue-green algae, are a natural source of active ingredients with unique properties.

Low-molecular-weight glycolipid extracts from cyanobacteria are well known to contain a lipid component belonging to the class of fatty acid esters of glycerol, for example monogalactosyl diacylglycerols (MGDG), digalactosyl diacylglycerols (DGDG), sulfoquinovosyl diacylglycerols (SQDG), phosphatidyl glycerol (PG), etc. (Murakami, et al., 1991, Chem. Pharm. Bull., 39: 2277-81).

The lipid membrane extracts with such composition, prepared from various Cyanobacteria, have shown various types of biological activities, for example anti-inflammatory properties as described, for instance, in EP1571203; and anti-tumoral properties as described in Shiraashi H., et al., 1993, Chem. Pharm. Bull., 41: 1664-66); sulpholipids extracted from Cyanobacteria also showed anti-HIV activity (Gustafson, J. Natl. Cancer Instit., 1989, 81:1254-1258). The authors of this patent application have previously described (Macagno A., et al., 2006, J. Exp. Med. 203:1481-92) that a crude extract from cells of Oscillatoria Planktothrix contains a LPS-antagonist activity directed to the TLR4 receptor, one of the main routes for recognition and entry of microbial pathogenic structures into cells responsible for immune recognition and stimulation (Rosenberger C. M. et al., 2003, Nat. Rev. Mol. Cell. Biol. 4:385-96).

It was subsequently reported that the anti-TLR4 activity is exerted also towards the LPS of Neisseria Meningitidis (Jemmett K, et al., 2008, Infect. Immun. 76:3156-63).

In fact, microbial proinflammatory stimuli act mainly through Toll-like receptors present on dendritic cells and macrophages, which translate pro-inflammatory stimuli through the nuclear factor NFkB, which in turn induces transcription of pro-inflammatory cytokines.

While this finding is extremely important to prevent the onset of inflammation of microbial origin, through inhibition of the binding of bacterial LPS to its own receptor, it has only limited impact on the extent of activation of the pro-inflammatory cascade which has already been initiated.

In fact, as it is well known, the process of inflammation (characterized by rubor, calor, tumor, dolor), which may also have microbial etiology, is triggered by a variety of stimuli, such as those arising from traumatic tissue damage or caused by tumors, UV light or hypoxia. In general, the response to an acute pro-inflammatory stimulus activates diverse intra- and extra-cellular signaling pathways exerting various effects, including altered permeability of capillary vessels which induces chemotaxis and affects cell adhesion through coordinated and redundant mechanisms leading to signal amplification. As result, inflammation may subside or, alternatively, such mechanisms may degenerate leading to chronic inflammation and fibrosis of the surrounding tissues.

Among commonly used, broad-spectrum anti-inflammatory therapies, it is worth to mention glucocorticoid treatment, involving osteoporosis and coagulation inhibition as side effects, and the treatment with non-steroidal anti-inflammatory drugs (NSAIDs), including more recently synthesized anti-COX-2 compounds, leading to decreased synthesis of vascular prostacyclin and therefore to an apparently higher cardiovascular risk.

Cytokine-specific anti-inflammatory strategies (such as treatment with inhibitors, e.g. antibodies capable of neutralizing TNF) also have limitations: in addition to being unable to reduce globally the pro-inflammatory response, which is highly redundant, they have various contraindications, including an increased risk for infections, e.g. latent and opportunistic infections, and unpredictable adverse effects in patients with heart disease or familiar demyelination.

Therefore in the field it would be highly desirable to discover non-steroidal anti-inflammatory drugs capable of acting on the central mediators which are activated early in the pro-inflammatory response.

It is now well recognized that necrotic cells are one of the most efficient pro-inflammatory stimuli and together with the release of an important mediator, extracellular ATP, a charged molecule of 650 Da which, upon cellular damage, efficiently crosses the aqueous intercellular space to bind and activate specific receptors (purinergic receptors present on a wide diversity of cell types). The extracellular ATP level is actively and specifically modulated also by enzymes present on the plasma membrane or in the extra- or peri-cellular environment (such as synthases, ecto-nucleotidases, etc.) involved in the mechanism that regulates the pro-inflammatory response. Therefore an increasingly consolidated hypothesis is that one of the most effective pro-inflammatory stimuli is extracellular ATP, both the pre-synthesized form released in the extracellular space upon cellular damage and the form synthesized de novo in the surrounding tissues during the early stages of inflammation (Di Virgilio F., 2007, Purinergic signal. 3:1-3; La Sala A, et al., 2003. J. Leukoc. Biol. 73:339-43). Enzymes, such as synthetases and ecto-nucleotidases, which concur to regulate extracellular ATP levels, are essentially ubiquitous proteins in the organism, unlike Toll-like receptors whose expression is restricted to immune cells, endothelial cells and adipocytes. In particular, the ATP synthase enzyme is expressed on many different cell types including hepatocytes, neurons, astrocytes, fibroblasts, immune cells, endothelial cells and is expressed at particularly high level on the plasma membrane of tumor cells (Mowery Y M, et al., 2008. Cancer Biol.& Ther.7:1836-38).

Therefore, detection of binding to the enzyme complex and of inhibition of ATP-synthase (ATP-SX) by the mixture purified according to the present invention, and detection of additive or possibly synergistic effects in reducing the more general inflammatory response via the previously observed anti-LPS activity, is extremely important in diseases where inflammation is not triggered by a microbial stimulus (e.g. autoimmune diseases, asthma, rheumatoid arthritis, psoriatic arthritis, Crohn's disease, multiple sclerosis and systemic vasculitis) (FIG. 1).

It is important to note that molecules with anti-inflammatory activity, like angiostatin, resveratrol and piceatannol, belong to a group of molecules capable of regulating the ATP-SX activity (Chavakis T. et al., 2005, Blood, 105:1036-43; Ashikawa K. et al., 2002, J. Immunol. 169:6490-7).

As shown in detail in the description of the present invention, the glycolipid mixture from Oscillatoria Plankothotrix is characterized by the presence of high molecular weight glycolipids of which the main component is a rhamnose-containing glycolipid. Rhamnose-containing glycolipids (rhamnolipids), with molecular weight below 10 KDa, have already been described in literature but have very different characteristics and properties from those of the present mixture.

For example, there is knowledge of a rhamnolipid isolated from the culture medium of Pseudomonas aeruginosa, a pathogenic bacterium for humans (Yokota et al. Eur. J. Biochem., 1987 167, 203-209). These substances can have biosurfactant activity (Zhang Y. and Miller R. Appl. Environ. Microbiol, 1994, 60:2101-2106). Furthermore, in EP 771191, a rhamnolipid was described that is released into the culture medium by a strain of Pseudomonas aeruginosa and has a cytotoxic activity in eukaryotic cells and is used for the treatment of autoimmune diseases.

### SUMMARY OF THE INVENTION

According to a first aspect, the invention relates to a mixture of polar glycolipids including, as major lipid component, at least one major fatty acid, whose length is comprised between C14 and C20, associated with a saccharide comprising at least one unit of rhamnose or its derivatives. This major glycolipid component has a molecular weight greater than 30 kDa as determined by polyacrylamide gel electrophoresis (PAGE).

The major glycolipid component includes stearic acid (C18:0, octadecanoic) and palmitic acid (C16:0, hexadecanoic), in an amount comprised for the former (C18:0, octadecanoic) between 50% and 80%, more preferably between 65% and 75% of the total lipid fraction and for the latter (016:0, hexadecanoic) between 15% and 40%, more preferably between 20% and 32%. Additional minor lipid species may be present in amounts not exceeding 15% of the total lipid fraction. The saccharide fraction of the glycolipid mixture consists of rhamnose or its derivatives in an amount of at least 20% of the total saccharide component, which, according to a preferred embodiment, includes also glucose or its derivatives and at least one additional saccharide or derivative selected from: xylose, mannose, galactose, galacturonic acid or their derivatives.

The mixture, suitably formulated under the form of compositions, has broad spectrum anti-inflammatory and immunomodulatory activity useful in acute and/or chronic inflammatory states which are induced by specific (e.g. microbial infections) as well as non-specific stimuli. However, it is particularly useful in the inflammation induced by non-specific stimuli, i.e. those not associated with a proven source of infection, such as inflammation associated with ischemic events, burns, severe trauma, hypoxic states, cancer, autoimmune diseases (such as multiple sclerosis, inflammatory bowel disease (Crohn's disease, ulcerative colitis), psoriasis, rheumatoid arthritis, diabetes, autoimmune thyroiditis, systemic lupus erythematosus) and, in general, in the inflammation associated with oxidative and/or nitrosylation stress or in systemic inflammatory states such as systemic inflammatory syndrome (SIRS), sepsis, vasculitis, or in localized inflammatory states, such as neurodegenerative diseases or asthma.

According to a further aspect, the invention relates to pharmaceutical compositions also for veterinary use, comprising the glycolipid mixture according to the invention as the active ingredient, in combination with suitable excipients and/or diluents. One particular aspect of the present invention relates to compositions for treatment of the inflammatory state associated with tumors and the pro-invasive effect of tumors, wherein the mixture is used along with a second active ingredient consisting of an anti-tumor agent and/or an immunosuppressant.

According to a further aspect, the invention relates to a method for preparation of polar glycolipids from extracts of the cyanobacterium Oscillatoria Planktothrix sp. (CCAP No. 1459/45), characterized in that said method involves a nuclease treatment step performed in order to obtain a level of nucleic acid contamination equal to or lower than 3% of the total weight, and a step of molecular separation using a device with a cutoff of 30 KDa, with recovery of the polar glycolipid fraction with greater molecular weight.

### BRIEF DESCRIPTION OF THE FIGURES

FIG. 1. Schematic example of regulatory mechanisms in the pro-inflammatory response, involving Toll-like receptor 4 (TLR4) and ATP synthase (ATP-SX) membrane enzyme.

FIG. 2 a) 1D NMR spectrum of the OPFP1 mixture. FIG. 2 b) Example of a chromatogram (GC-MS) of the sugars detected in the glycolipid mixture.

FIG. 3. Mono-dimensional electrophoresis analysis (DOC-PAGE) of the mixture according to the invention. Panel A: Lane 1: E. coli LPS (serotype 0111:B4) 10 μg; Lane 2: mixture 7 μg, Lane 3: mixture 3.5 μg.

FIG. 4. Labeling of the neuroblastoma line SH-SY5Y a) with the OPFP1 mixture coupled to the fluorochrome Alexa Fluor 555; b) with E. coli LPS coupled to the fluorochrome FITC.

FIG. 5. SDS-PAGE of plasma membrane proteins extracted from the neuroblastoma cell line SH-SY5Y and bound to the magnetic beads coupled to the OPFP1 mixture. Lane 1: MW standard; lane 2: cell lysate+beads without mixture; Lane 3: cell lysate+beads coupled to the OPFP1 mixture. Arrows indicate ATP synthase α, β, γ subunits and their respective molecular weights.

FIG. 6. Production of extracellular ATP. Measurement of the effect of OPFP1 on the production of extracellular ATP in the SH-SY5Y cell line. (A) analysis of the effects on ATP production at different pre-incubation times; (B) evaluation of the effects elicited by different concentrations of the OPFP1 mixture (results are the mean of three independent experiments).

FIG. 7. Inhibition with the OPFP1 mixture of the production of pro-inflammatory cytokines induced by E. coli LPS in a human monocytic cell line (THP1).

FIG. 8. Inhibition with the OPFP1 mixture of the production of pro-inflammatory cytokines induced by P. gingivalis LPS in a human monocytic cell line (THP1).

FIG. 9. Inhibition with the OPFP1 mixture of the TNF-alpha production induced by PMA in a human monocytic cell line (THP1). Comparison with a mixture obtained as described in Macagno A. et al., 2006, J. Exp. Med. 203:1481-92.

FIG. 10. Effect of the OPFP1 mixture on cell survival after dopamine induction of oxidative stress in a human dopaminergic cell line (SH-SY5Y). Symbol legends: -▴-: OPFP1 20 μg/ml; --; OPFP1 10 μg/ml; -▪-: medium only.

FIG. 11. Inhibition of tumor invasion in Matrigel® by the OPFP1 mixture at a concentration of 10 μg/ml. Melanomas: SKMEL and 9923M; carcinomas: HEY4 and HEY3MET7.

FIG. 12. Inhibition of total PMA-induced MMP-9 production in presence of the OPFP1 mixture at a concentration of 10 μg/ml in the following cell lines: 9923M, HEY4 and HEY3MET7. Gray bars: 10⁻⁷M PMA; black bars: 10⁻⁷M PMA+OPFP1 10 μg/ml.

### DETAILED DESCRIPTION OF THE INVENTION

### Definitions

Saccharide, possibly substituted: includes monosaccharides, disaccharides, oligosaccharides in which the glycosidic portion may show the presence of aglycone-type substituents like amino acids, phosphate and sulfate charged groups.

Glycolipid: molecules composed of carbohydrates, including saccharides (sugars) or their polymers, associated with lipid or fatty acid chains or covalently linked to them by amide or ester bonds.

Lipid chain (fatty acid): aliphatic monocarboxylic acids. Long-chain fatty acids may be saturated, unsaturated or hydroxylated.

Rhamnolipid: glycolipid whose lipid fraction comprises at least one fatty acid chain and whose saccharide fraction consists of at least one unit of rhamnose or its derivatives.

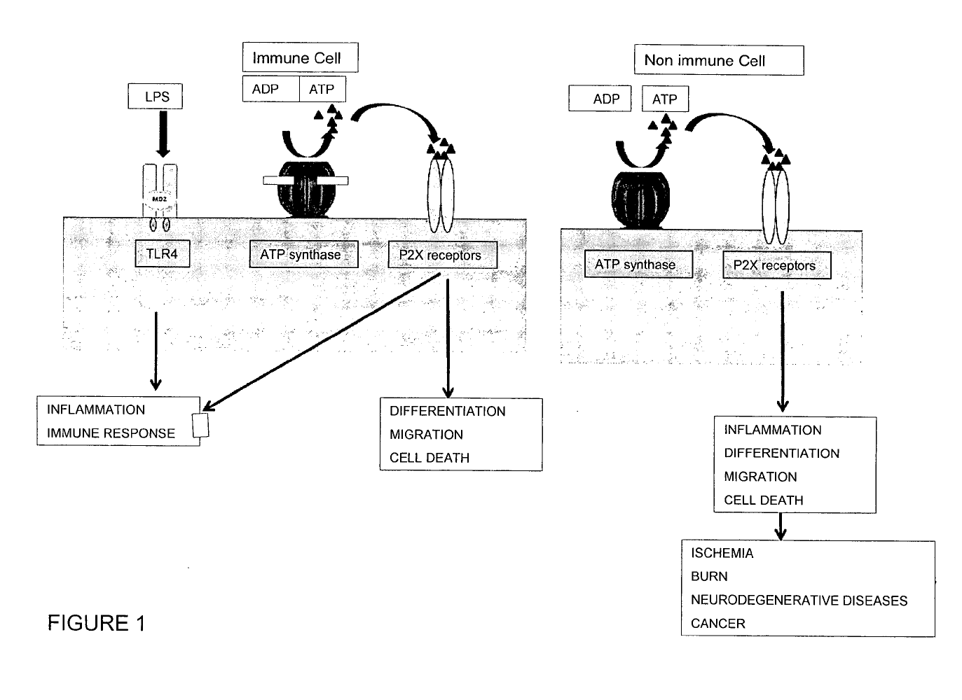

The inventors of the present application have found that a highly purified preparation of polar glycolipids, extracted from the cells of the cyanobacterium Oscillatoria Plankothotrix sp. (CCAP No. 1459/45) and designated “OPFP1 mixture” for the purpose of the present invention, is characterized by the presence of at least one high-molecular-weight glycolipid species comprising one or more rhamnose units. Within such mixture, characterized by the absence of protein contaminants, as assessed, for example, by the Bradford method and characterized by a nucleic acid contamination lower than or equal to 3%, it was possible to detect an inhibitory activity toward ATP synthase (ATP-SX). Such activity is capable of decreasing the inflammatory response separately from the antagonistic activity toward bacterial LPS binding via the specific TLR4 receptor (Macagno A., et al., 2006, J. Exp. Med. 203:1481-92; Jemmet K et al., 2008, Infect. Immun. 76:3156-63) which, on its own, stimulates the pro-inflammatory response. This effect shows a synergistic character due to the redundancy and overlap of the pathways signaling the pro-inflammatory stimulus in cells that possess both the TLR4 receptor and the membrane ATP synthase activity, as for instance monocytes, adipocytes or endothelial cells (FIG. 1). The ATP synthase inhibitory activity is associated with major components of the glycolipid mixture, which is devoid of the typical keto-deoxyoctulosonic acid (KDO) reactivity characteristic of LPS from Gram-negative bacteria, has a molecular weight equal to or greater than 30 kD, as detected for example by PAGE and silver-staining, and accounts for up to 70-90% of the purified mixture. The OPFP1 mixture comprises stearic acid (C18:0, octadecanoic) and palmitic acid (C16:0, hexadecanoic) as fatty acids of the major glycolipid component. Such fatty acids exist preferably in their saturated form and they are preferably present in amounts respectively comprising (where 100 is the total lipid fraction) 50%-80% stearic acid and 15%-40% palmitic acid, each in respect to the total lipid fraction of the glycolipid mixture, and even more preferably such fatty acids are present in amounts comprised between 65-75% and 20-32%, respectively. Other fatty acids chains may be present in amounts not exceeding 15% and preferably comprise Lauroleic acid (or dodecenoic acid (C12: 1)) in amounts not exceeding 10%, preferably not more than 5% of the lipid fraction. The analysis of fatty acids can be carried out by GC-MS after hydrolysis of the compound with methanol-hydrochloric acid (HCl 1M/MeOH) for 20 hours at 80° C. and extraction as methyl esters in the hexane phase, as better described in Experimental Part. As noted above, the major glycolipid component is characterized in that it comprises at least one unit of rhamnose or its derivatives, and by molecular weights greater than 30 kDa, preferably between 30 and 40 kDa, which can be determined, for example, by DOC-PAGE followed by staining with silver salts. The glycolipid mixture according to the present invention is devoid of phospholipids and free lipids, which are eliminated by the extraction procedure. Moreover, the glycolipid mixture according to the present invention is also devoid of low molecular weight glycolipids such as fatty acid derivatives of glycerol (monogalactosyl diacylglycerols (MGDG), digalactosyl diacylglycerols (DGDG), sulfoquinovosyl diacylglycerols (SQDG), which are typical of cyanobacteria) Thylakoid membranes, have a molecular weight generally lower than 1 kDa and are discarded in the procedure of the present invention. The analysis of sugars of the glycolipid mixture indicates that, within the saccharide component (given as 100%), rhamnose is preferably 20-40%, glucose (Glc) is 20-40%, xylose (Xyl) is 5-10%, mannose (Man) is 3-5%, galactose (Gal) is 3-5%, glucosamine (GlcN) is 0.5-1%, galacturonic acid (GalA) is 1-6%, where these sugars can be simple sugars or more preferably they are complex sugars and/or sugar derivatives. Sugars like 2-keto-3-deoxyoctonate (KDO), typical of bacterial (Gram-negative) LPS are not detected. Qualitative and quantitative analysis of the saccharide residues can be done by GC-MS analysis of acetylated methyl glycosides after hydrolysis of the glycolipid mixture with methanol-hydrochloric acid (HCl 1M/MeOH) for 20 hours at 80° C., extraction in hexane and acetylation with pyridine and acetic anhydride and/or by analysis of alditol acetates obtained after treatment with 2M trifluoroacetic acid for 1 hour at 120° C., followed by treatment for 1 hour with sodium borohydride and subsequent acetylation with pyridine and acetic anhydride. To the inventors' knowledge, there is no previous description of a mixture of high-molecular-weight, polar glycolipids extracted from cyanobacterial cells and characterized by the presence of rhamnose, that is absolutely non-toxic to mammalian cells. Moreover the presence of an anti-inflammatory activity which is capable of targeting also the inflammation induced by the above defined non-specific stimuli was never identified in known rhamnolipids. In fact, the present glycolipid mixture acts by a mechanism identified for the first time in the present invention, owing to the high degree of purification that has been achieved, in particular by removing nucleic acids. Indeed the immunostimulatory activity of nucleic acids, a process mediated by specific Toll-Like-Receptors, is well known. Unlike the already-known LPS-antagonist activity (induced by microbial components), which is exerted via the TLR4-MD2 receptor, such a mechanism is activated by interaction with the primary cellular receptor for rhamnolipid, a substance associated with the main component of the glycolipid mixture, and leads to direct inhibition of the membrane ATP synthase enzyme. Therefore the activity of the present glycolipid mixture is defined as broad spectrum anti-inflammatory activity targeting inflammation induced by both microbial stimuli and non-specific stimuli. Binding studies performed with the purified mixture on TLR4-negative cells (e.g. the SH-SY5Y neuroblastoma line) and on a monocytic cell line (THP-1), expressing both the TLR4 receptor and high levels of membrane ATP-SX, have shown that such mixture interacts with subunits of the ATP synthase F1 complex present on the plasma membrane of different cell types, thereby inhibiting production of extracellular ATP. Indeed the complex comprising the glycolipid and the ATP-SX subunit has been isolated by “affinity binding” and electrophoresis and characterized by LC-ESI-MS/MS mass spectrometry, as better detailed in the Experimental Part. Due to the key role played by extracellular ATP, the modulation of ATP-SX activity by OPFP1 causes an overall decrease of the inflammatory response that is much higher than the reduction obtained entirely via the already-known LPS antagonist activity, as it is better detailed in the experimental part of the present application. Without being bound to any theory, this finding is plausibly due to the fact that pro-inflammatory pathways activated by very different stimuli, both specific and non-specific, such as bacterial LPS or PMA, act through shared mediators and regulatory molecules. Thus the mixture of polar glycolipids of the present invention acts by a dual mechanism: not only as antagonist of Gram-negative bacterial LPS, via interaction with the TLR4-MD2 receptor complex (although lacking structural similarities to LPS), but mainly by reducing immune and inflammatory responses through inhibition of extracellular ATP production (a mechanism independent from the interaction with the TLR4-MD2 complex, hence from direct cellular stimulation by microbial components). Molecules capable of regulating the ATP-SX activity are, for example, angiostatin, resveratrol and piceatannol, which possess anti-inflammatory activity (Chavakis T. et al., 2005, Blood, 105:1036-43; Ashikawa K. et al., 2002, J. Immunol. 169:6490-7). Using the human monocytic cell line THP-1, it has been also observed that the administration of the mixture, at concentrations of 1 and 10 μg/ml, can decrease the production of pro-inflammatory cytokines, like TNF-alpha, which is activated by a non-LPS-specific pro-inflammatory stimulus, such as Phorbol Myristate Acetate (PMA). As better described in detail in the Experimental part, detection of such activity is made possible by the high degree of purity of the mixture. Since extracellular nucleotides, especially extracellular ATP, act as “natural adjuvants” inducing a series of inflammatory-like activities (mediated by autocrine and paracrine interactions of ATP with purinergic receptors present on antigen presenting cells (APC, macrophages, dendritic cells) (La Sala A., et al., 2003, J. Leukoc. Biol. 73:339-43), it is now clear that this inhibitory mechanism (that is independent from the interactions with microbial components) is of primary importance for regulation of the immune response.

Reduced availability of ATP in the extracellular microenvironment is important in order to inhibit the inflammatory response in the early phase and is directly linked to the therapeutic efficacy of the compound, in addition to its LPS-antagonist activity, which is mainly preventive. Unlike pure TLR4 receptor antagonists (Manthey C. L., et al. 1993, Infect. Immun. 61:3518-26), the mixture acts by inhibiting production of inflammatory cytokines even at a 1:1 concentration ratio with bacterial LPS and even after the administration of LPS.

Following administration of the OPFP1 mixture according to the invention, at an optimal concentration of 10 μg/ml, the level of extracellular ATP can be reduced to 63%, as assessed by use of an in vitro system lacking TLR4-MD2, like SH-SY5Y human neuroblastoma cells, in which such effect can be detected in absence of any contamination. After having found that the mixture, prepared as described in Example 1, is capable of antagonizing the biological effects of all types of LPS, i.e. those acting exclusively via the Toll-like-receptor 4 (TLR4) and those acting via interaction with TLR4 and TLR2 (Darveau R P, et al. 2004, Infect. Immunity 72:5041-51) and, most importantly, that such mixture can counteract non-specific pro-inflammatory stimuli, it can be concluded that the OPFP1 mixture can inhibit production of pro-inflammatory cytokines regardless of the type of stimulus used to induce their production. In addition, the observation that extracellular ATP plays a key role in inflammatory processes involving cells other than those directly involved in the immune response (macrophages and dendritic cells) has been verified also in neuronal cells, particularly during the process of cell death in response to stimuli such as oxidative stress. The effectiveness of the mixture in the models presented in the experimental part (in vitro reduction of apoptosis induced by dopamine in neuronal cells and reduction of kainic acid-induced seizures) confirmed that the OPFP1 mixture inhibits apoptosis in neuronal cell lines and is capable of preventing the underlying mechanism of neurodegeneration. The scientific literature confirms a close link between sporadic neurodegenerative diseases and oxidative and nitrosylation stress, glycosylation, inflammatory mechanisms and the consequences of persistent high levels of excitatory neurotransmitters, which are considered among the major risk factors. Currently employed therapies are essentially symptomatic, with variable effectiveness depending on disease and the state of the individual patient. Further confirmation of the effectiveness of such application is provided by the recently published observation that high levels of extracellular ATP induce death of dopaminergic neuronal cell lines (Jun D-J et al. J. Biol. Chem., 2007, 282, 37350-8). Among other things, the lack of toxicity, even at rather high concentrations, makes the OPFP1 mixture suitable not only for treatment of inflammatory diseases, or diseases with a predominant inflammatory response, but also for treatment of neurodegenerative diseases. In particular, the OPFP1 mixture, and compositions comprising OPFP1 as active ingredient, have a broad spectrum anti-inflammatory and immunomodulatory activity which is useful in acute or chronic inflammatory states induced by specific stimuli (e.g. microbial infections by gram-positive bacteria, gram-negative bacteria, viruses or parasites such as yeasts or fungi) as well as in acute or chronic inflammatory states caused by non-specific stimuli. However, it is noted that the present mixture and its compositions are particularly useful in inflammation induced by non-specific stimuli, i.e. those not associated with a proven source of infection, such as, for example, inflammation associated with ischemic events (in particular cerebral ischemia), burns, severe trauma, hypoxic states, cancer, autoimmune diseases (such as multiple sclerosis, inflammatory bowel disease (Crohn's disease, ulcerative colitis), psoriasis, rheumatoid arthritis, diabetes, autoimmune thyroiditis, systemic lupus erythematosus) and, in general, in inflammation associated with oxidative and/or nitrosylation stress or in systemic inflammatory states such as systemic inflammatory syndrome (SIRS), sepsis, vasculitis, or in localized inflammatory states, such as neurodegenerative diseases or asthma. Among neurodegenerative diseases, the treatment of syndromes associated with neuronal cell death, such as Parkinson's disease, Alzheimer's disease, amyotrophic lateral sclerosis, epilepsy, senile dementia and Hungtington corea, is preferred. In addition, experimental data indicate that the OPFP1 mixture is also capable of specifically inhibiting Type 9 matrix metalloproteinase (MMP-9): therefore, use of the mixture of the invention is claimed in order to limit the process of metastasis, preferably in combination with the appropriate anti-cancer therapy. The anti-metastatic activity added to the above described anti-inflammatory activity, makes the use of the mixture according to the invention particularly beneficial for treatment of invasive cancers with an important inflammatory component. Moreover, the treatment of infections with the mixture according to the invention may be particularly indicated in patients with heart damage or familiar demyelination, in which treatment with cytokine-specific anti-inflammatory agents (such as treatment with inhibitors, for example TNF-neutralizing antibodies) is not appropriate. The complete water solubility of the compound makes possible a therapeutic use in pharmaceutical compositions with aqueous excipients and diluents, which are well known to the expert in the field, especially with respect to liquid formulations. According to a further aspect, the mixture is also suitable for preparation of compositions for veterinary use, with the same therapeutic purpose described above. The treatment of veterinary inflammatory diseases resulting from microbial infections (caused by Gram-positive bacteria, Gram-negative bacteria, viruses or parasites such as yeasts or fungi or components thereof), or resulting from invasive cancer, is particularly preferred. According to a further aspect, the present invention relates to a process for preparation of a mixture of polar glycolipids, wherein the major component has a molecular weight above 30 kDa, derived from a cyanobacterium, in particular from Oscillatoria Planktothrix FP1 (No 1459/45, CCAP Culture Collection of Algae and Protozoa, SAMS Research Services Ltd., Dunstaffnage Marine Laboratory, Dunbeg, ArgyII, PA37 1QA, UK). In this process, the bacterial pellet is first treated with denaturing agents, such as a mixture of organic solvents and chaotropic agents, for example a mixture of phenol and guanidinium thiocyanate, and with deproteinizing agents; the process is characterized by treatment with nucleases until reaching a level of nucleic acid contamination lower than or equal to 3% of the total weight, preferably lower than or equal to 2%, and by a step of molecular separation on a device with a cutoff of 30 KDa, followed by recovery of the higher molecular weight fraction. According to a further detail, cyanobacteria are grown in culture medium, for example BG-11 medium (Sigma-Aldrich, Cat No. C3061), at a temperature of 25° C. and under cool-white light at a light intensity of 5 umol. m⁻².sec⁻¹ and in a continuous 24-hour light regimen.

Upon reaching the stationary growth phase, the cyanobacterial culture is sedimented, for example by centrifugation (pelletting); the sediment (or pellet) may be frozen or freeze-dried prior to extraction. After thawing or rehydration (in the case of freeze-drying), the sediment is processed through the following steps: a) the pellet is resuspended by dilution preferably with a volume of water or aqueous solvent comprised between 1:1 and 1:2; b) to obtain a cell extract containing the glycolipid mixture (also termed supernatant), the resuspended pellet is mixed, as described above, with an appropriate volume of solution containing denaturing agents, for example as described in Chomczynski P. and Mackey (Biotechniques, 1995, 19(6):942-5), preferably comprising a reagent based on a polar protic organic solvent, preferably phenol, and a chaotropic agent (such guanidinium thiocyanate), as for instance the Trireagent® (Sigma cat. N T3934) or similar reagents like Trizol® (Invitrogen), and an aprotic organic solvent such as chloroform, at a ratio of about 1 volume of aqueous suspension of cyanobacteria, 2-4 volumes of extraction solution, preferably about 3 volumes, and about 0.5-1 volume of chloroform; c) the cell extract is incubated for at least 5 minutes, more preferably for a time length of at least 10 minutes but not exceeding 60 minutes, at room temperature; d) centrifugation, preferably at approximately 2000×g, and the supernatant (aqueous phase) containing the polar glycolipid fraction is collected. Such supernatant can be measured, for example, by electrophoresis and silver-staining or by biological activity assays, like inhibition of TNF-alpha production in the monocyte/macrophage derived THP1 cell line in presence of LPS, or the binding to ATP synthase (ATP-SX) or the inhibition of its activity. The pellet obtained by centrifugation in d) can be re-extracted by adding water or an aqueous buffer (in an amount approximately equal to the volume collected), followed by re-centrifugation of the sample. This second supernatant is combined with the previous supernatant, and the pooled sample is then subjected to further steps involving: e) precipitation by addition of salt, e.g. sodium acetate (5-20 mM final) and of an organic solvent, preferably acetone, in an amount of about 2 volumes, followed by centrifugation, under the same conditions as above, and by pellet washing with ethanol diluted in water, e.g. 70% ethanol, at least once and preferably twice, f) resuspension of the pellet in a preferably buffered aqueous solution, for instance 50 mM TRIS. An enzymatic treatment of nucleic acid contaminants is then carried out by use of endo- and/or exo-nucleases (e.g. DNase and RNase), followed by enzymatic treatment of protein contaminants by digestion with a protease, e.g. proteinase K, preferably at 100 μg/ml for sufficient time (at least 1 hour at 37° C.). After enzymatic digestion (step g), the sample is again centrifuged, the supernatant is collected and further precipitated by addition of salt (e.g. sodium acetate, about 10 mM final) and of an appropriate volume (preferably 2 volumes) of organic solvent, preferably acetone, centrifuged again, followed by resuspension of the pellet in water or aqueous solution containing at least one surfactant agent, preferably deoxycholate (DOC), and also preferably containing a cationic surfactant like tetraethylammonium; the material is again extracted with the (denaturing) extraction solution described in section b), and then is further precipitated with sodium acetate/acetone as described above, washed with 70% ethanol, resuspended in water or in aqueous solution, and subjected to molecular separation by use of a filter (or other suitable device) with a cut off of 30 KDa, thus eliminating the material that passes through the filter and recovering the higher molecular weight glycolipid fraction in the retentate, in water or buffered aqueous solution, with a level of nucleic acid contamination lower than 3%.

Moreover, elemental analysis performed on the OPFP1 mixture showed the presence of carbon (39%), hydrogen (6.2%), nitrogen (5.8%), sulfur (0.5%). According to a further aspect, the invention comprises a polar glycolipid mixture obtainable from the cyanobacterium Oscillatoria Planktothrix FP1 (No. 1459/45, CCAP) according to the above described method, wherein the major glycolipid component is a rhamnolipid with molecular weight equal to or greater than 30 KDa and with broad-spectrum anti-inflammatory activity.

### EXPERIMENTAL PART

### Example 1

### Preparation of the Glycolipid Mixture from Cyanobacteria

Oscillatoria Planktothrix cyanobacteria FP1, CCAP repository No. 1459/45 (deposited on Sep. 7, 2008 in the Center for Culture Collection of Algae and Protozoa, Scotland, UK) were collected by centrifugation from the BG-11 culture medium (Cyanobacteria BG-11 freshwater solution cat. N. C3061, Sigma Aldrich). Collected cyanobacterial cells were frozen and thawed, diluted 1:2 with water, mixed with 3 volumes of Tri-reagent and 1 volume of chloroform and incubated for 10 minutes at room temperature. After incubation, cell debris were centrifuged at 2000×g for 15 min and the supernatant (aqueous phase), containing the active fraction, was recovered. The cyanobacterial cell pellet was further extracted by re-addition of water (an amount equal to the previously collected volume) and the sample was again centrifuged. Collected supernatants were precipitated with sodium acetate (10 mM final), 2 volumes of acetone and centrifuged. After centrifugation, the supernatant was removed and the pellet was further washed with 70% ethanol to remove membrane phospholipids. After removing the supernatant, the pellet was dissolved in 50 mM TRIS and 10 mM MgCl₂ solution, pH 7.5, containing DNase (20 μg/ml) and RNase (10 μg/ml). The sample was incubated at 40° C. for 2 hours, prior to addition of proteinase K (100 μg/ml) for overnight incubation at 37° C. The following day the sample was centrifuged at 2000×g for 15 min; the supernatant was recovered and precipitated with sodium acetate (10 mM final) and 2 volumes of acetone. The pellet obtained after centrifugation was resuspended in a solution containing sodium deoxycholate (0.5%) and tetra-ethyl-ammonium (0.2%), and then re-extracted with Tri-Reagent, according to the above described procedure. After precipitation with sodium acetate/acetone and washing in 70% ethanol, the sample was resuspended in water and subjected to a series of purifications by use of a ultrafiltration device (Amicon Ultra-15 centrifugal filter units Millipore cat. N. UFC 903008) with molecular weight cutoff of 30-kDa in order to remove low-molecular-weight contaminants, followed by final resuspension of the sample in water or buffered saline (PBS) for the subsequent chemical and biological tests.

### Example 2

### Analysis of the Glycolipid Mixture

The mixture, extracted as described in Example 1, is a preparation consisting mainly of high molecular weight polar glycolipids extracted from the cyanobacterium Oscillatoria Plankothotrix FP1. The preparative procedure that makes use of a washing step with 70% ethanol removes phospholipids from the mixture.

The absence of free lipids has been verified by Folch partitioning (chloroform/methanol/water 3:2:1).

The absence of low molecular weight glycolipids such as monogalactosyl diacylglycerols (MGDG), digalactosyl diacylglycerols (DGDG), sulfoquinovosyl diacylglycerols (SQDG), phosphatidyl glycerol (PG) and rhamnolipids (RL) was verified by TLC, a method that is specific for these glycolipids. In addition, the mixture is characterized by the absence of protein contamination detectable by the Bradford method and the presence of ≦3% nucleic acid contamination.

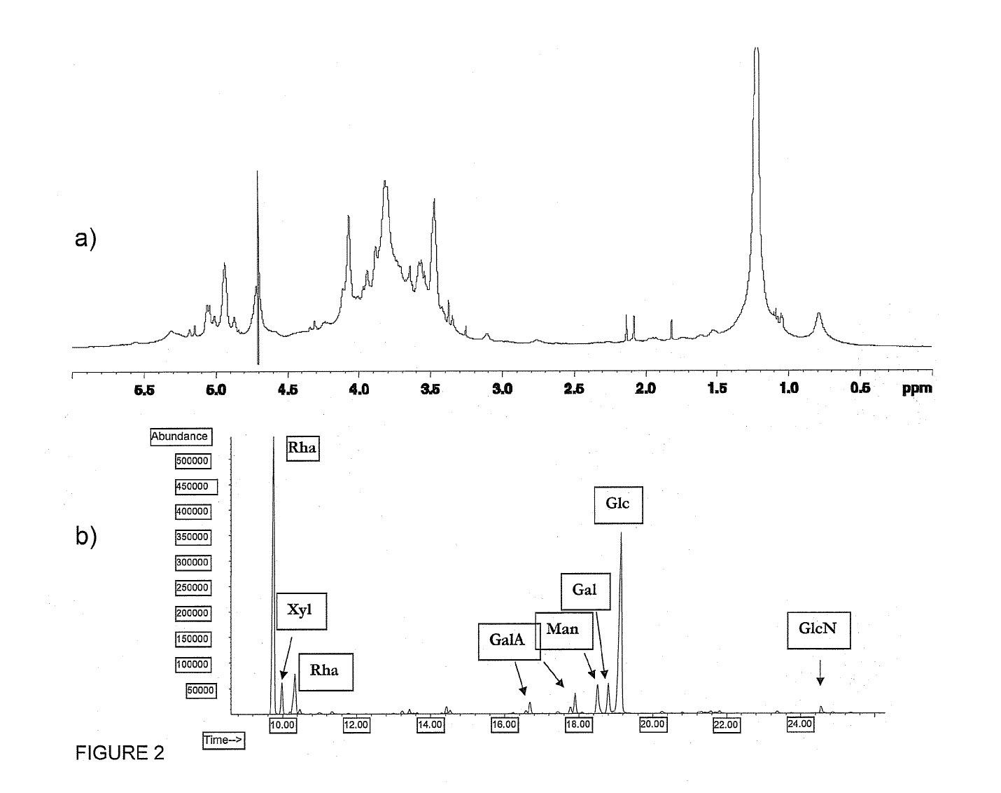

FIG. 2a shows the 1D NMR spectrum of the OPFP1 mixture. In the spectrum, signals detected in the region comprised between 4.5 and 5.5 ppm originate from the presence of different anomeric sugars. The intense high field signal (1.3 ppm) confirms the presence of deoxy-sugars in the mixture. The peak at 0.8 ppm identifies the presence of terminal CH₃ groups of the lipid chains.

Purified glycolipids are different from lipopolysaccharides (LPS) of Gram-negative bacteria due to complete absence of the characteristic sugar 2-keto-3-deoxyoctonate (KDO), as assessed by GC/MS analysis of monosaccharides (FIG. 2b). Moreover, as confirmed by 1D-³¹P NMR spectral analysis, glycolipids extracted from the cyanobacterium do not show the presence of phosphate groups, unlike LPS from Gram-negative bacteria. Qualitative and quantitative analysis of saccharide residues was performed by GC-MS analysis of acetylated methyl glycosides and alditol acetates; similarly, fatty acids were detected under the form of methyl esters by GC-MS through the analysis of the retention times of chromatographic peaks and fragmentation of mass spectra.

In detail, an aliquot of the sample (1 mg) was treated with methanol-hydrochloric acid (HCl 1M/MeOH) for 20 hours at 80° C. and subsequently essicated and extracted with hexane; the hexane phase contains fatty acids under the form of methyl esters, while the methanol phase contains O-methyl glycosides.

Acetylated methyl-glycosides were prepared as follows: the methanolic phase was essicated under air flow and acetylated with 50 μl of pyridine and 50 μl of acetic anhydride at 100° C. for 30 min. The mixture was essicated, dissolved in CHCl₃, extracted several times with water in order to purify the sample, and then recovered and essicated.

For alditol acetates, an equal aliquot of sample was treated with 2M trifluoroacetic acid at 120° C. for 1 hour. After the acid was essicated, the sample was dissolved in water and treated for 1 hour with one spatula-tip of sodium borohydride. Excess hydride was destroyed with acetic acid and the solution was essicated several times with methanol and acetic acid. Finally, acetylation was carried out as for acetylated methyl glycosides.

Both acetylated methyl glycosides and alditol acetates were analyzed by GC-MS (Gas Chromatography-Mass Spectrometry). The results of the analysis of acetylated methyl glycosides and alditol acetates can be viewed in the chromatograms, where each derivatized monosaccharide shows its own retention time; for each peak, a typical mass spectrum is associated with each monosaccharide. The area under the peaks is proportional to the amount of monosaccharide present in the mixture. The analysis of alditol acetates makes possible to confirm and complete the panel of the monosaccharides detected, with the additional advantage to provide an individual signal for each monosaccharide. The following sugars were found to be present (FIG. 2b): Rhamnose (Rha) 39.4%; Glucose (GLc) 38%; Xylose (Xyl) 9.6%; Mannose (Man) 4.2%; Galactose (Gal) 3.9%; Glucosamine (GlcN) 1%; Galacturonic acid (GalA) 2%.

The lipids obtained after treatment of the compound with methanol-hydrochloric acid and extraction with hexane, for separation from methyl glycosides, were analyzed by GC-MS, which yielded the following fatty acid composition: C12:1 (lauroleic acid or dodecenoic acid) 3.1%; C16:0 (palmitic acid or hexadecanoic acid) 27.4%; C18:0 (stearic acid or octadecanoic acid) 68.7%.

The elemental analysis performed on the mixture showed the presence of carbon (39%), hydrogen (6.2%), nitrogen (5.8%), sulfur (0.5%).

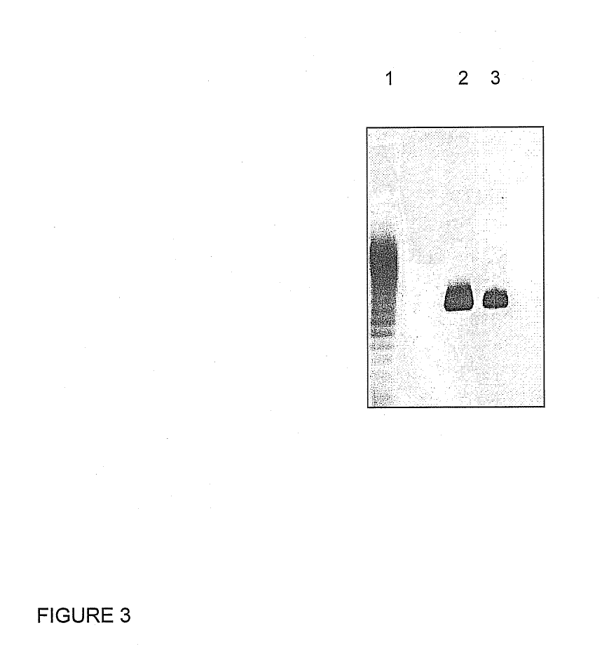

A single main band, with molecular weight around 30 kDa, is observed by electrophoresis and silver staining, a specific method for display of high-molecular-weight glycolipids that makes use of sodium deoxycholate (DOC-PAGE) as detergent that promotes electrophoretic mobility of the compound and helps disaggregating micellar structures formed in presence of high concentrations of glycolipids in aqueous solution (FIG. 3).

The glycolipid sample is soluble in water; the solution is clear, colorless, odorless, tasteless; highly concentrated glycolipids aggregate to form micelles. The sample is stable even after boiling for 5 min or after one free-thaw cycle.

### Example 3

### Identification of the Membrane Receptor

To identify the plasma membrane receptor which mediates pharmacological effects, the mixture was conjugated to a fluorescent dye, and various human cell lines were used as targets for in vitro labeling experiments detected by fluorescence microscopy. In particular, cell lines derived from human melanoma (SKMEL-28), ovarian cancer (HEY4), neuroblastoma (SH-SY5Y) and an embryonic kidney epithelial cell line (HEK293) were tested (Molteni et al., Cancer Letter, 2006, 235:75-83). The glycolipid mixture was treated with sodium periodate (to oxidize the saccharidic component, thereby introducing a functional aldehyde group), incubated for 30 min at room temperature with Alexa 555 fluorochrome (Molecular Probe) and was finally incubated with sodium borohydride (1 mM). At the end of the incubation, the labeled glycolipid mixture was precipitated with sodium acetate/acetone and finally resuspended in water. For labeling experiments on various cell lines, cultures were prepared on glass slides; after fixation, slides were incubated with the labeled glycolipid mixture, washed and visualized by fluorescence microscopy using a FITC filter.

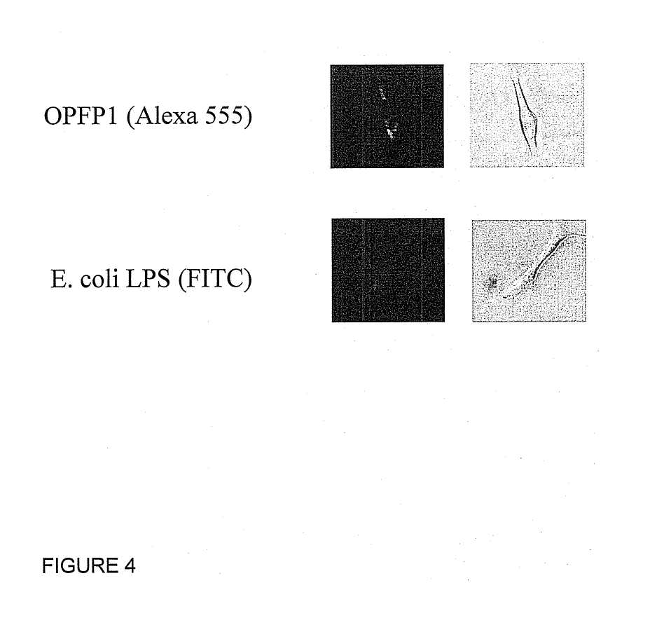

Results showed that the glycolipid mixture positively labeled all cells except those of embryonic origin (HEK293). A labeling example is shown in FIG. 4: it is possible to observe that the neuroblastoma line SH-SY5Y is positively labeled with the glycolipid mixture conjugated to Alexa Fluor 555, whereas does not bind the fluorescently labeled bacterial LPS (LPS-FITC Sigma), therefore indicating that the plasma membrane receptor for LPS (TLR4-MD2) is absent. Because of these characteristics, the SH-SY5Y cell line was used for identification of an alternative receptor different from the TLR4-MD2 complex (Molteni et al. 2006, see above). After biotinylation, the mixture was conjugated with magnetic beads carrying surface streptavidin (Promega cat. N.Z5481) (0.4 mg of biotinylated mixture for 1.8 ml magnetic beads). Plasma membrane proteins obtained from 30×10⁶ SH-SY5Y cells were extracted in their native form by use of a Calbiochem kit, ProteoExtract Cat. N. 444810, incubated with plain magnetic beads (to remove proteins that are physiologically biotinylated) and then with magnetic beads conjugated with the OPFP1 glycolipid mixture. Proteins specifically bound to OPFP1-conjugated beads were subjected to one-dimensional electrophoresis under denaturing conditions, stained with Coomassie and analyzed by LC-ESI-MS/MS mass spectrometry.

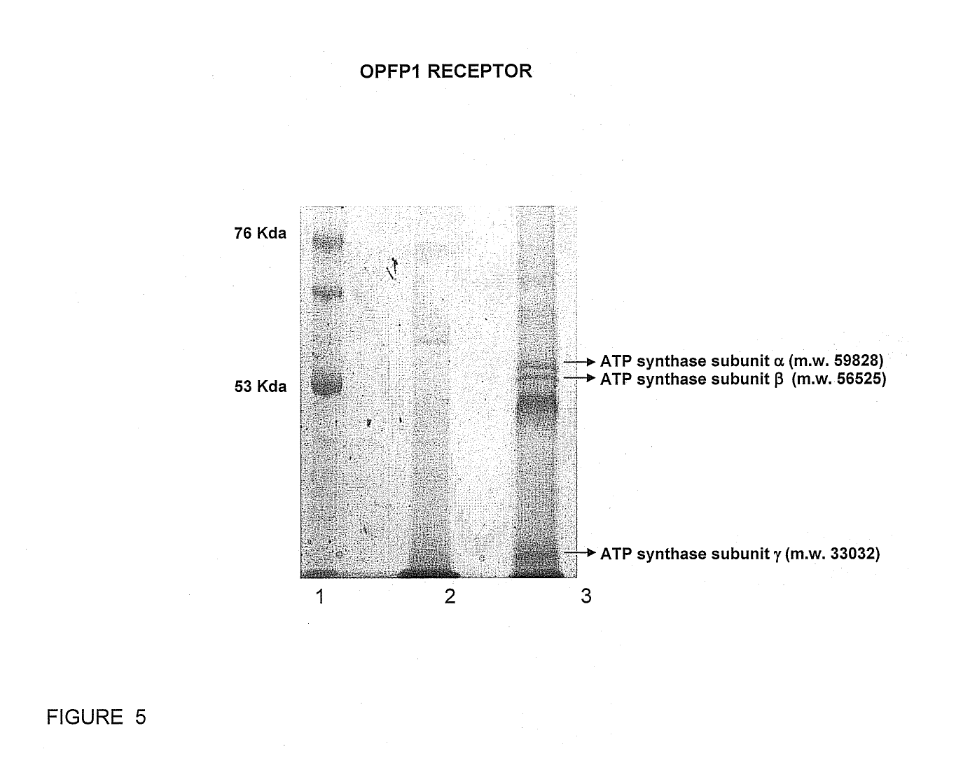

The sequencing data revealed that the plasma membrane proteins that specifically interact with the purified preparation are α, β, γ subunits of the human ATP synthase complex (FIG. 5). These results were also confirmed with the human monocytic line THP1 which expresses the ATP synthase complex on the cell surface, in addition to the TLR4-MD2 receptor.

The mixture not only binds this enzyme complex but also modulates its activity. As positive control, piceatannol (3,4,3′,5′-tetrahydroxy-trans-stilbene) was used, that is a resveratrol metabolite well-known for its inhibitory activity on the production of extracellular ATP.

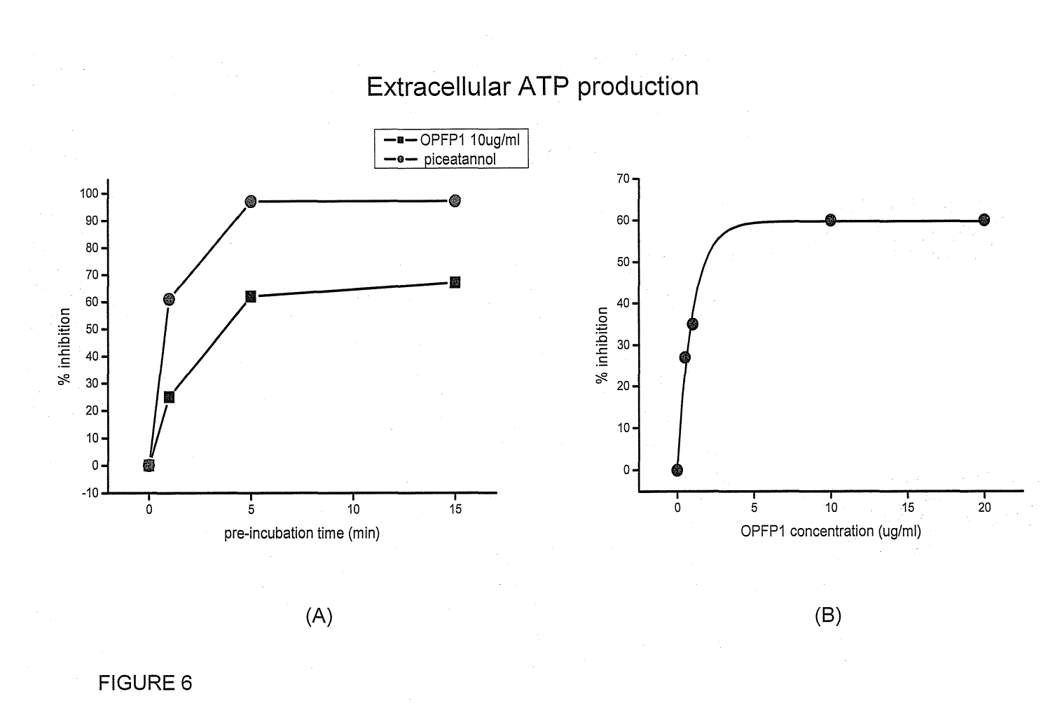

The SH-SY5Y cell line was then pre-incubated in vitro with the OPFP1 mixture (10 μg/ml) or piceatannol (4 μM) for various times (1′, 5′, 15′); after 5′, the maximum inhibition of ATP synthesis was 63% for the mixture, and 97% in presence of piceatannol (FIG. 6A). Additional experiments carried out with different concentrations of OPFP1 (0.5, 1, 10, 20 μg/ml) showed that the inhibition is dose-dependent and is maximal at a concentration of 10 μg/ml (FIG. 6B). The results show that the purified material not only binds membrane ATP synthase but also inhibits its activity in a dose-dependent manner.

### Example 4

### Inhibition of Pro-Inflammatory Cytokines Induced by Different Stimuli in a Human Monocytic Cell Line

The mixture prepared as described in Example 1 was used to study the effects in vitro on production of pro-inflammatory cytokines in a human monocytic cell line (THP1) expressing both plasma membrane TLR4-MD2 complex and ATP synthase. Both bacterial LPS and nonspecific stimuli (such as Phorbol Myristate Acetate, PMA) were used for cellular activation.

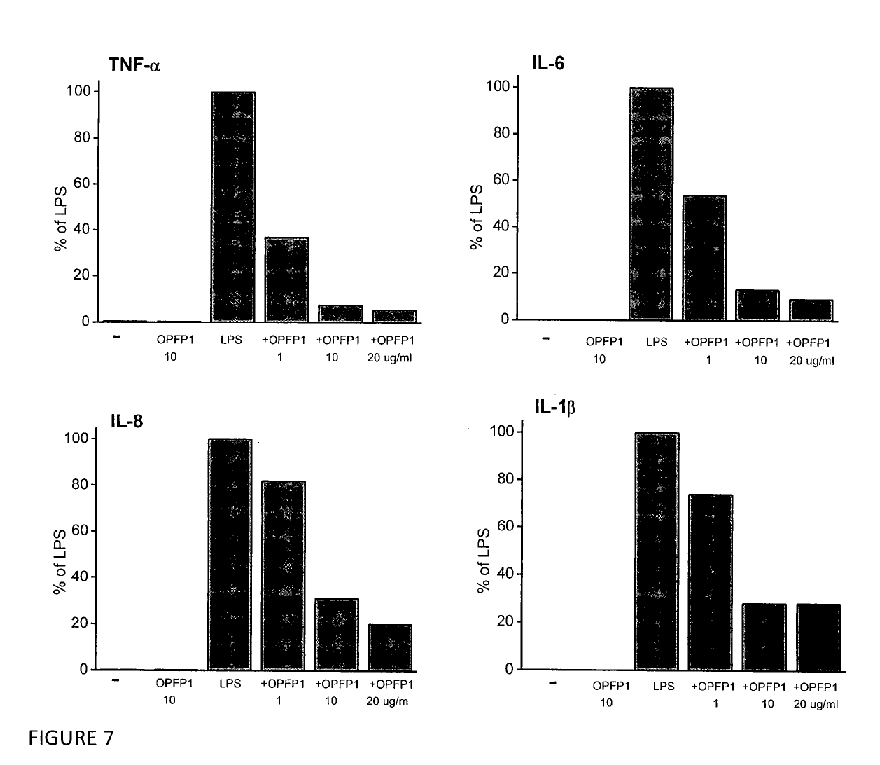

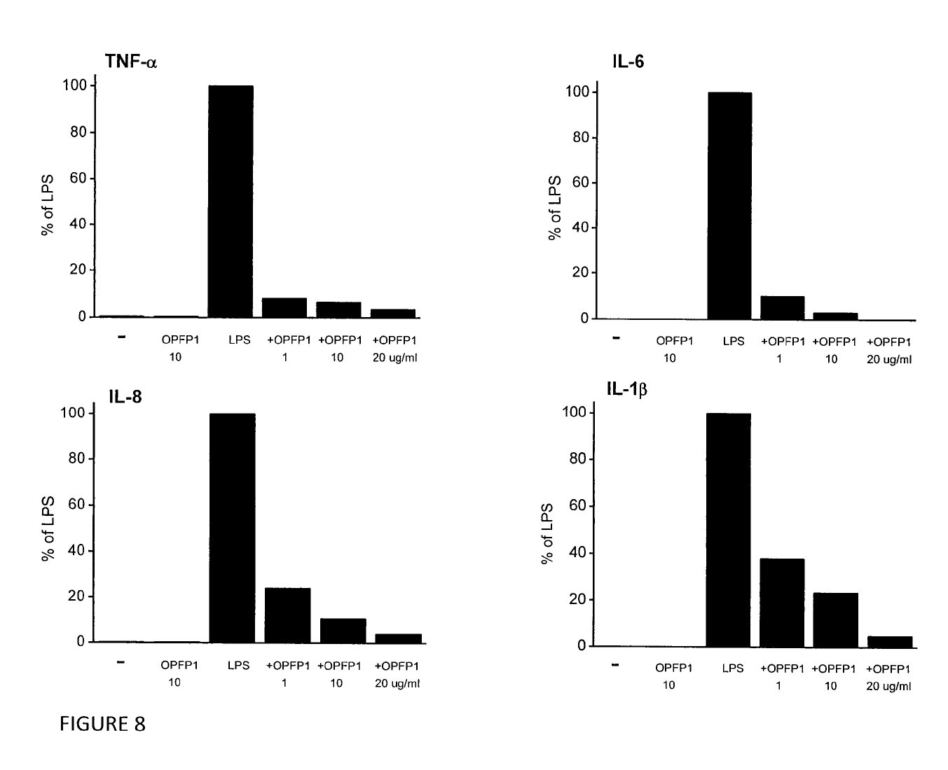

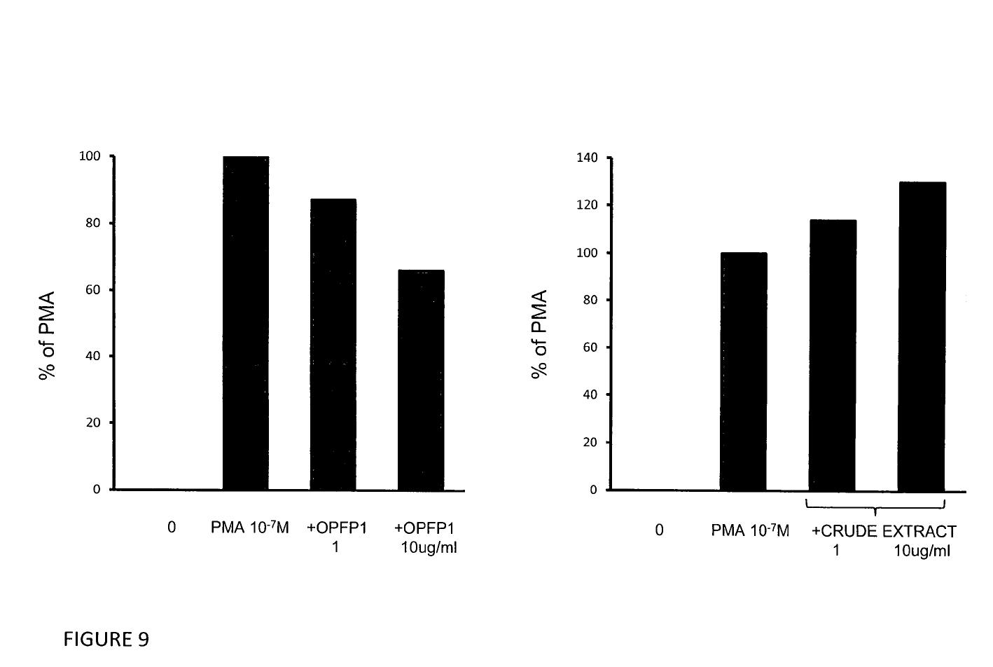

The results confirmed that the mixture does not stimulate production of pro-inflammatory cytokines in culture (FIG. 7) and showed that, in presence of bacterial LPS (from both E. coli and P. gingivalis, respectively, in the experiments described in FIGS. 7 and 8), which stimulate cytokine production, the mixture acts as an antagonist, thereby inhibiting production of tumor necrosis factor alpha (TNF-alpha), interleukin 6 (IL-6), interleukin 1 beta (IL-1 beta), interleukin 8 (IL-8), in a dose-dependent manner. Inhibition of cytokine induction by LPS from Escherichia coli serotype 0111:B4 is shown by data in FIG. 7, while FIG. 8 shows the results obtained in experiments with LPS from Porphyromonas gingivalis. It is well known that E. coli LPS is a pure TLR4 agonist, while P. gingivalis LPS is agonist for both TLR4 and TLR2 (Darveau R P, et al., 2004, Infectimmunity 72:5041-51). The regulation of TNF-alpha production was also studied in presence of PMA. Results shown in FIG. 9 indicate that only the highly pure glycolipid mixture acts as inhibitor of TNF-alpha production even in presence of a nonspecific stimulus like PMA, hence via a pathway that is independent from the mechanism activated in presence of LPS. Indeed, inhibition is not observed in presence of a crude extract from the same cyanobacterium, obtained according to the method described. Thus, after having observed that the mixture prepared as described in Example 1 can antagonize all LPS types, i.e. those acting exclusively through TLR4 and those acting via interaction with TLR2, in addition to antagonizing non-specific pro-inflammatory stimuli, it is possible to conclude that the OPFP1 mixture is capable of inhibiting production of pro-inflammatory cytokines, regardless of the type of stimulus used to induce their production.

Data obtained from stimulation of THP1 cells with E. coli or P. gingivalis LPS allows to assert that inhibition of production of pro-inflammatory cytokines by inhibition of membrane ATP synthase and antagonism at the level of TLR4 receptor are certainly additive and possibly synergistic effects. Indeed we note that, in presence of a sub-optimal concentration of the inhibitory glycolipid mixture, i.e. 1 μg/ml, the inhibitory capacity of the mixture is much higher in cultures incubated with P. gingivalis LPS, which acts via both TLR4-dependent and TLR4-independent stimulatory activities, than in cultures incubated with E. coli LPS. Thus, the additional inhibitory activity is due to effects other than TLR4 receptor antagonism, and must therefore be ascribed to the inhibition of membrane ATP-SX. The inhibition of pro-inflammatory cytokines due to inhibition of membrane ATP-SX can be estimated from the difference between the percentage of inhibition by P. gingivalis LPS and E. coli LPS, as follows:

| | E. coli LPS | P. gingivalis LPS | |
|---|---|---|---|
| | (1 μg/ml) | (1 μg/ml) | Difference |
| Cytokine | % inhibition | % inhibition | (ATP-SX activity) |
| TNF-alpha | 62% | 93% | 31% |
| IL1-beta | 26% | 58% | 32% |
| IL-6 | 46% | 93% | 47% |
| IL-8 | 18% | 78% | 60% |

From the results obtained with stimulation of production of pro-inflammatory cytokines by LPS species which are not exclusively TLR4 receptor agonists, it is clear that ATP-SX inhibition concurs, by a mechanism of synergistic interaction, to inhibit production of pro-inflammatory cytokines; ATP-SX inhibition contributes by 30%-60% to the total inhibitory effect, depending on the pro-inflammatory cytokine considered. Moreover, to fully evaluate the role played by ATP-SX, the efficiency of inhibition of TNF-alpha production in presence of the glycolipid mixture extracted from the cyanobacterium was compared in vitro with a typical pure TLR4 antagonist, the LPS from Rhodobacter sphaeroides (Invivogen). The results showed that R. sphaeroides LPS, added to the culture medium at a concentration 20-fold higher than E. coli LPS, is unable to inhibit TNF-alpha production, while the glycolipid mixture from the cyanobacterium almost completely inhibits TNF-alpha production (mean inhibition 97%) at a concentration 20-fold higher than E. coli LPS. The data on inhibition obtained with the PMA stimulus are consistent with what has been shown for other membrane ATP synthase inhibitors, e.g. piceatannol (Ashikawa K., et al., 2002, J. Immunol, 169:6490-7), well-known to inhibit production of pro-inflammatory cytokines induced by LPS and PMA by this mechanism.

### Example 5

### Inhibition of Dopamine-Induced Apoptosis in SH-SY5Y Neuroblastoma Cells

To assess the effects of the mixture in cell lines of neuronal origin, a known system was used (Colapinto M. et al. Biochem. Biophys. Res Commun., 2006, 349, 1294-300) in which dopaminergic cell lines (for example the SH-SY5Y neuroblastoma line, whose membrane ATP synthase is regulated by the OPFP1 glycolipid mixture, as shown in FIGS. 5 and 6) were treated with dopamine in vitro to induce cellular apoptosis. Incubation with dopamine makes possible to increase its intracytoplasmic levels, thereby mimicking cellular damage caused by free radicals and reactive intermediates produced in neurons during detoxification from dopamine itself. Dopamine-induced apoptosis is the model used to study phenomena involved in degeneration of dopaminergic neurons, as observed in Parkinson's disease.

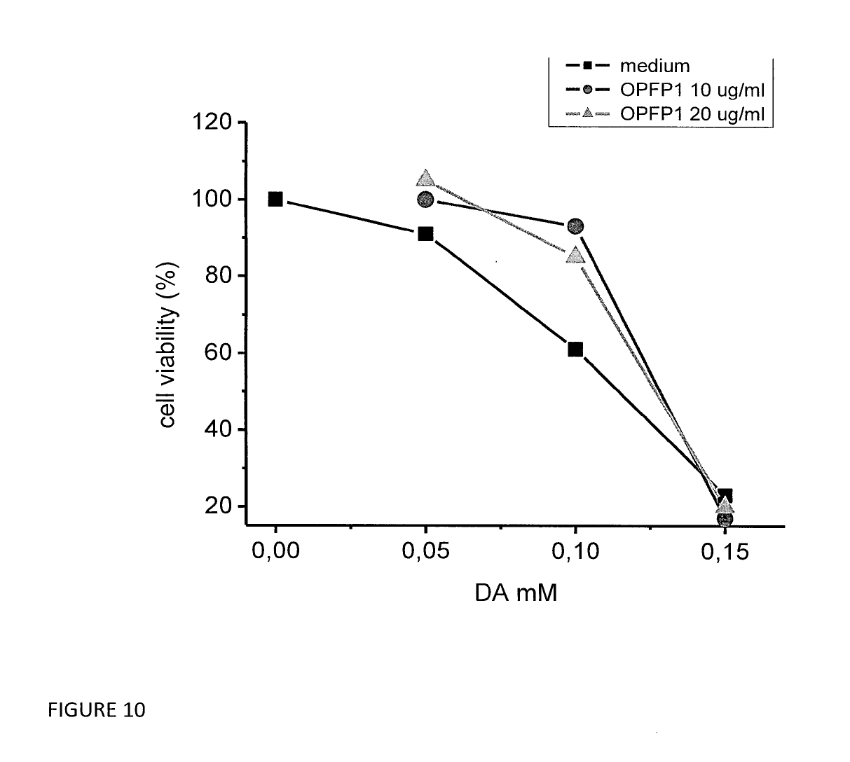

To this end, the SH-SY5Y neuroblastoma cell line was treated with different concentrations of dopamine (0.05-0.1-0.15 mM) for 24 hours, in presence or absence of the purified material at final concentrations of 10 and 20 μg/ml. Dopamine induced apoptosis in dose-response manner, with a mean survival of 91%, 61%, 23% at dopamine concentrations of 0.05, 0.1, 0.15 mM, respectively. An improvement of cell survival was observed in presence of the mixture. In particular, at 20 μg/ml concentration of the mixture, cell survival was 100%, 85%, 20% in presence of 0.05, 0.1, 0.15 mM dopamine, respectively (FIG. 10).

The neuroprotective activity was further confirmed in vivo in a murine model in which seizures were reproduced by induction with kainic acid. The purified mixture was able to reduce the epileptic activity by 40%.

Without being committed to any particular theory, the usefulness of the mixture in this experimental model is apparently confirmed by an observation made by other research groups, according to which extracellular ATP plays a fundamental role in the inflammatory processes associated with neurodegeneration.

### Example 6

### Inhibition of Tumor Invasion in Matrigel® and Inhibition of the Production of Metalloproteinase 9 in Human Tumor Cell Lines

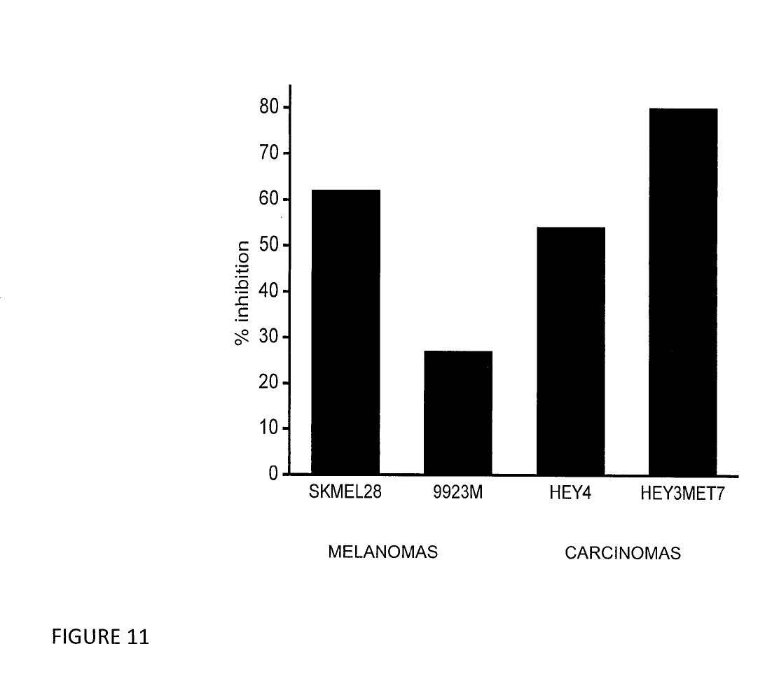

It is well known that membrane ATP synthase inhibitors such as angiostatin and resveratrol derivatives have anti-tumor effects (Tabruyn S. P., et al., 2007, Biochem Biophys. Res. Commun. 350, 1-8; Kundu J. K., et al., 2008, Cancer Lett. 269:243-61). Thus, the mixture according to the present invention was first evaluated in an in vitro proliferation test and subsequently in an inhibition assay for tumor cell invasiveness in Matrigel®. In the latter assay, several melanoma and carcinoma tumor cell lines derived from primary tumors or metastases, in particular SKMEL-28 (human melanoma line), 9923M (melanoma line from lymph nodal metastasis), HEY4 (ovarian carcinoma line), HEY3-MET7 (metastatic ovarian carcinoma line derived from HEY4 after injection into nude mice, described in Molteni et al. Cancer Letter 2006, see above), were subjected to a chemotactic stimulus represented by fetal bovine serum, using a Boyden chamber where the pores of the septum that separates the upper from the lower chamber are occluded with a matrix made of gelatinous protein mixture resembling extracellular environment, termed Matrigel® (QCMEC Matrix Cell Invasion Assay cat. N. ECM550 Chemicon International). Cells were seeded in the upper chamber in medium lacking fetal calf serum, in presence or absence of the glycolipid mixture (at a concentration of 10 μg/ml), whereas the complete culture medium containing serum was added to the lower chamber. After incubation at 37° C. for 24 hours in 5% CO₂ atmosphere, the number of cells invading the lower chamber, following lysis of the Matrigel®, was estimated by crystal violet staining. Results presented in FIG. 11 indicate that the glycolipid mixture, purified as in Example 1 and used at a concentration of 10 μg/ml, inhibits the ability of tumor cells to migrate and invade, under basal conditions, by 30% up to a maximum of 80%, depending on the cell line tested.

Tumor invasiveness is a very complex process involving the release of enzymes, such as gelatinases (MMP-2 and MMP-9) which degrade the extracellular matrix thereby allowing tumor cells to reach the bloodstream and migrate at distance from the site of primary tumor. A study on the total release of MMP-9 in some cell lines showed that the mixture inhibits migration in Matrigel® because can significantly inhibit MMP-9 production. In a preliminary phase, the release of gelatinase into the culture medium was qualitatively evaluated by zymography only in some cell lines (SKMEL-28 and HEY4) (data not shown), while subsequent quantification of MMP-9 was performed by ELISA (MMP-9 Biotrak activity Assay® cat. N. RPN2634 Amersham Bioscences). Preliminary zymography made possible to find out that SKMEL-28 cells do not produce MMP-9, neither under basal conditions nor after PMA stimulation, but only produce MMP-2 constitutively, and that MMP-2 levels are also inhibited by the OPFP1 glycolipid mixture.

In contrast, zymography performed on HEY4 cell culture supernatants only showed the presence of MMP-9, whose levels are increased after incubation with PMA.

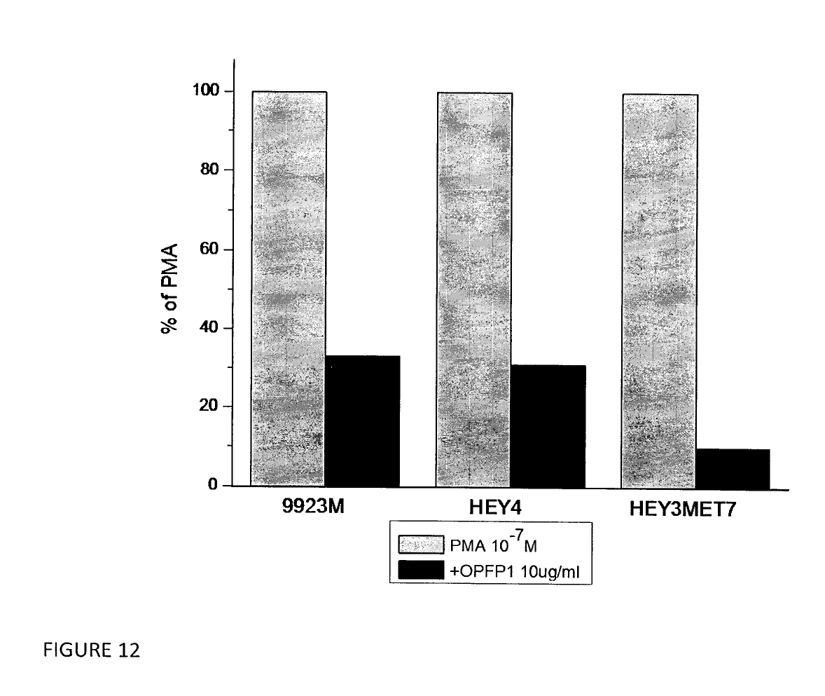

FIG. 12 shows the results of stimulating MMP-9 release by 10⁻⁷M PMA, in presence or absence of the OPFP1 glycolipid mixture at a concentration of 10 μg/ml, in three of the above cell lines, during incubation at 37° C. in 5% CO₂ atmosphere for 24 hours; the results show a significant inhibitory effect of the glycolipid mixture of the invention on MMP-9 production, which is essential for tumor cell migration during the metastatic process.

1. A mixture of polar glycolipids comprising stearic acid (C18:0, octadecanoic) and palmitic acid (C16:0, hexadecanoic) as fatty acids of the major glycolipid component, wherein one of them is associated with a saccharide comprising at least one unit of rhamnose or its derivatives.

2. Mixture according to claim 1 wherein stearic acid is in an amount comprised between 50% and 80%, more preferably between 65% and 75%, and palmitic acid (C16:0, hexadecanoic) is in an amount comprised between 15% and 40%, more preferably between 20% and 32%, wherein said major glycolipid component has a molecular weight higher than 30 kDa.

3. Mixture according to claim 2 wherein the lipid fraction comprises in addition a fatty acid different from stearic acid and palmitic acid, in an amount not exceeding 15% of the total lipid fraction.

4. Mixture according to claim 3 wherein at least one of said fatty acid different from stearic acid and palmitic acid, is lauroleic acid (C12:1, dodecenoic).

5. Mixture according to claim 4 wherein rhamnose, or its derivatives, is present in amounts of at least 20% of the total saccharide component of the glycolipid mixture.

6. Mixture according to claim 5 wherein the total saccharide component also comprises glucose or its derivatives.

7. Mixture according to claim 6 wherein said saccharide component comprises in addition at least one saccharide selected from the group consisting of: xylose, mannose and galactose and galacturonic acid or derivatives thereof.

8. A method of preparing a medicament, the method comprising formulating the mixture according to claim 1 for pharmaceutical use.

9. Mixture A method of treating inflammation in a subject, the method comprising administering to the subject a mixture according to claim 1.

10. A method for the treatment of acute and/or chronic, systemic or organ-specific inflammatory diseases with a microbial etiology and/or of non-specific nature in a subject, the method comprising administering to the subject a mixture according to claim 1.

11. The method according to claim 10, wherein said inflammatory diseases of non-specific nature is associated with ischemia, burns, severe trauma, hypoxia, cancer, oxidative and/or nitrosylation stress, Graft versus Host Disease, or systemic or organ-specific autoimmune disease.

12. The method according to claim 11, wherein said autoimmune disease is selected from the group consisting of: multiple sclerosis, inflammatory bowel disease, Crohn's disease, ulcerative colitis, psoriasis, rheumatoid arthritis, diabetes, autoimmune thyroiditis and systemic lupus erythematosus.

13. The method according to claim 11 wherein said inflammatory disease is selected from the group consisting of: systemic inflammatory syndrome (SIRS), sepsis, neurodegenerative disease associated with cell death, Parkinson's disease, Alzheimer's disease, amyotrophic lateral sclerosis, epilepsy, senile dementia, Huntington corea, systemic vasculitis, and asthma.

14. A composition comprising as active ingredient the mixture according to claim 1, in combination with suitable excipients and/or diluents.

15. Composition according to claim 14 for veterinary use.

16. Method for preparing a polar glycolipid mixture from cells of the cyanobacterium Oscillatoria Planktothrix sp (CCAP No. 1459/45), said polar glycolipid mixture comprising: stearic acid (C18:0, octadecanoic) and palmitic acid (C16:0, hexadecanoic) as fatty acids of the major glycolipid component, wherein at least one of them is associated with a saccharide comprising at least one unit of rhamnose or its derivatives, said method comprising the steps of:
extracting with denaturing chaotropic agents;
treating with nucleases until reaching a level of nucleic acid contamination lower than or equal to 3% of the total weight; and
molecular separating on a device with a cutoff of 30 Kda, followed by recovering the polar glycolipid mixture from the higher molecular weight fraction.

17. Method according to claim 16 wherein the extraction step is performed as described in step b), nuclease treatment is performed as described in step g), molecular separation is performed as described in step k) and comprises the further steps a), c)-f), h) j), and l)-m):
a) resuspension of a concentrate of the cyanobacterium Oscillatoria Planktothrix sp. (No. 1459/45), optionally also freeze-dried, with an aqueous solution in a volume ratio preferably comprised between 1:1 and 1:2;
b) mixing the suspension of cyanobacteria with 2-4 volumes, preferably about 3, of a denaturing solution comprising a chaotropic agent, a polar protic organic solvent, preferably phenol, and an aprotic organic solvent such as chloroform,
c) incubation for a time shorter than 60 minutes;
d) centrifugation and collection of the liquid phase, termed supernatant;
e) precipitation of the glycolipid fraction by addition of a salt and an organic solvent, preferably acetone, to the supernatant and washing the precipitate (or pellet) with water-diluted ethanol;
f) resuspension of the precipitate in an aqueous solution, preferably buffered;
g) treatment with nucleases, preferably endo- and/or exo-nucleases and subsequently with a protease, preferably proteinase K;
h) precipitation of the glycolipid phase by addition of a salt and an organic solvent, preferably acetone;
i) optional step of pellet washing with water-diluted ethanol and resuspension in aqueous solution comprising ionic surfactants, preferably sodium deoxycholate, and a quaternary ammonium salt;
j) re-extraction from the aqueous solution as described in steps b)-f);
k) molecular separation on a device with a cutoff of 30 KDa;
l) recovery of the high molecular weight polar glycolipid fraction with water or buffered aqueous solution and assessment of the level of nucleic acid or protein contamination;
m) recovery and use of the fraction having less than 3% nucleic acid contamination.

18. Glycolipid mixture obtained from a culture of the cyanobacterium Oscillatoria Planktothrix sp. (No 1459/45) according to the method of claim 17 wherein the major glycolipid component is a rhamnolipid with molecular weight higher than or equal to 30 kDa with broad-spectrum anti-inflammatory activity.

19-23. (canceled)
